(19) United States
(12) Patent Application Publication
(10) Pub. No.: US 2016/0066610 A1
(43) Pub. Date: Mar. 10, 2016

(54) METHODS FOR ENHANCING AGED MUSCLE REGENERATION

(71) Applicant: ABBOTT LABORATORIES, Abbott Park, IL (US)

(72) Inventors: SEAN GARVEY, Columbus, OH (US); SUZETTE PEREIRA, Westerville, OH (US); NEILE EDENS, Austin, TX (US)

(73) Assignee: ABBOTT LABORATORIES, Abbott Park, IL (US)

(21) Appl. No.: 14/888,314

(22) PCT Filed: May 1, 2014
(86) PCT No.: PCT/US2014/036297

§ 371 Date: Oct. 30, 2015

Related U.S. Application Data
(60) Provisional application No. 61/818,237, filed on May 1, 2013.

(51) Int. Cl. A23L 1/30; A61K 31/19; A61K 31/353; A23L 1/305; A23L 1/29

(52) U.S. Cl. CPC A23L 1/3002; A23L 1/305; A23L 1/296; A23L 1/3051; A61K 31/353; A61K 31/19; A23V 2002/00

(57) ABSTRACT

Compositions and methods for enhancing the regenerative capacity of an individual are provided. The compositions include, and the methods provide, a combination of an effective amount of epigallocatechin-3-gallate (EGCg) and an effective amount of beta-hydroxy-beta-methylbutyrate (HMB) to decrease the level of intramuscular FGF2, to enhance the regenerative capacity of muscle, or both.

### CROSS REFERENCE TO RELATED APPLICATIONS

This application claims priority to and the benefit of U.S. Provisional Application No. 61/818,237, filed May 1, 2013, the entire content of which is incorporated herein by reference.

### TECHNICAL FIELD

The general inventive concepts relate to methods for improving muscle function, and more particularly to the use of an effective amount of both EGCg and HMB to reduce the amount of intramuscular FGF2, to improve the regenerative capacity of muscle, or both.

### BACKGROUND

In general, the ability of a muscle to regenerate is directly tied to muscle mass and muscle function. Thus, when muscle loses its ability to effectively regenerate, muscle loss and loss of muscle function often follow. A decline in muscle function can have a number of adverse effects on an individual including, but not limited to, general weakness, fatigue, a lessening of joint mobility, a reduction in physical activities, vulnerability to falls, and a general decline in functional status.

Loss of muscle function (including loss of muscle mass) may occur from a number of factors and conditions including, age, disuse (including recovery from inactivity), as well as muscle wasting diseases, such as, cachexia due to cancer, end stage renal disease (ESRD), acquired immune deficiency syndrome (AIDS), or chronic obstructive pulmonary disease (COPD). Generally, these are associated with a loss of muscle mass, a decline in the ability of muscle to regenerate, or combinations thereof.

### SUMMARY

Compositions and methods for enhancing the regenerative capacity of muscle in an individual, and for reducing the level of intramuscular FGF2 are provided herein. The methods include administering a combination of active ingredients which, in combination, decrease the level of intramuscular FGF2 in an individual to achieve a desired therapeutic effect. Thus, the methods disclosed herein comprise administration of both epigallocatechin-3-gallate (EGCg) (or a source of EGCg) and beta-hydroxy-beta-methylbutyrate (HMB) (or a source of HMB) to an individual in need thereof in an amount effective to decrease the level of intramuscular FGF2, which in turn leads to an increase in the regenerative capacity of muscle. In certain embodiments, the methods include administering the EGCg and HMB as part of a nutritional composition.

In a first exemplary embodiment, a nutritional composition for enhancing the regenerative capacity of muscle in an individual is provided. The nutritional composition comprises a source of EGCg in an amount sufficient to provide 0.1 to 1 gram of EGCg per serving of nutritional composition; and a source of beta-hydroxy-beta-methylbutyrate (HMB) in an amount sufficient to provide 0.5 to 3 grams of HMB per serving of the nutritional composition.

In a second exemplary embodiment, a method for enhancing the regenerative capacity of muscle in an elderly individual is provided. The method comprises administering to an elderly individual, a nutritional composition comprising a therapeutically effective amount of epigallocatechin-gallate (EGCg), and a therapeutically effective amount of beta-hydroxy-beta-methylbutyrate (HMB). Administration of the nutritional composition results in an enhancement of regenerative capacity of the elderly individual's muscle(s).

In a third exemplary embodiment, a method for reducing the intramuscular level of fibroblast growth factor (FGF2) of an elderly individual is provided. The method comprises administering a nutritional composition comprising a therapeutically effective amount of epigallocatechin-gallate (EGCg) and a therapeutically effective amount of beta-hydroxy-beta-methylbutyrate (HMB) to an elderly individual, wherein the HMB and EGCg are present in the composition in a weight ratio between 1:2 to 30:1. Administration results in a decrease in the intramuscular level of FGF2 in the muscle(s) of the elderly individual.

While several exemplary embodiments are discussed in detail herein, still other embodiments of the general inventive concepts will become apparent to those skilled in the art from the following detailed description, which shows and describes exemplary embodiments of the invention. As will be realized, the invention is capable of modifications in various aspects, all without departing from the spirit and scope of the inventive concept.

### BRIEF DESCRIPTION OF THE DRAWINGS

FIG. 1 is a graph illustrating the results of an 8-week dietary supplementation study involving groups of aged (21 months) rats. The first group was fed a control diet, the second group was fed the control diet with added EGCg, the third group was fed the control diet with added HMB, and the fourth group was fed the control diet with both EGCg and HMB added thereto.

### DETAILED DESCRIPTION

The general inventive concepts include compositions and methods for enhancing the regenerative capacity of muscle in an individual, and methods for reducing the level of intramuscular FGF2. The methods include administering a combination of epigallocatechin-3-gallate (EGCg) (or a source of EGCg) and beta-hydroxy-beta-methylbutyrate (HMB) (or a source thereof) to an individual in need thereof in an amount effective to increase the regenerative capacity of muscle, reduce the level of intramuscular FGF2, or both. In certain embodiments, the methods include administering a nutritional composition containing EGCg and HMB.

The terminology as set forth herein is for description of the exemplary embodiments only and should not be construed as limiting the disclosure as a whole. Unless otherwise specified, “a,” “an,” “the,” and “at least one” are used interchangeably. Furthermore, as used in the description and the appended claims, the singular forms “a,” “an,” and “the” are inclusive of their plural forms, unless the context clearly indicates otherwise.

The term “nutritional composition” as used herein, unless otherwise specified, refers to nutritional compositions in various forms including, but not limited to, liquids, solids, powders, semi-solids, semi-liquids, nutritional supplements, and any other nutritional food product known in the art. A nutritional composition in powder form may often be reconstituted to form a nutritional composition in liquid form. In certain exemplary embodiments, the nutritional composition further comprises at least one source of carbohydrate, at least one source of protein, at least one source of fat, or combinations thereof. The nutritional compositions disclosed herein are generally suitable for oral consumption by a human.

The term “individual” as used herein, unless otherwise specified, refers to a mammal, including companion animals, livestock, laboratory animals, working animals, sport animals, and humans. In certain exemplary embodiments, the individual is a human.

The term “individual in need thereof” as used herein, unless otherwise specified, refers to an individual exhibiting muscle loss due at least in part to age (also referred to herein as an elderly individual), inactivity, injury, disease, or combinations thereof. In certain exemplary embodiments, the muscle loss in the individual in need thereof is at least partially attributable to increased muscle protein degradation, decreased muscle protein synthesis, decreased muscle regeneration, or combinations thereof. In certain exemplary embodiments, the individual in need thereof, is an elderly human, optionally a diseased elderly human. In certain exemplary embodiments, the individual in need thereof, is a human that is undergoing a temporary or permanent period of inactivity, due to disability, temporary injury or healing from an operation. In certain exemplary embodiments, the individual in need thereof is a human undergoing rehabilitation (i.e., physical rehabilitation) due to disease, injury, surgery, hospital admission, and combinations thereof. The term diseased, when referring to an individual in need thereof, refers to an individual with a muscle wasting disease. Non-limiting examples of muscle wasting diseases include cachexia due to cancer, end stage renal disease (ESRD), acquired immune deficiency syndrome (AIDS), and chronic obstructive pulmonary disease (COPD).

The term “elderly” as used herein, refers to an individual of at least 40 years of age, including at least 45 years of age, at least 50 years of age, at least 55 years of age, at least 60 years of age, at least 65 years of age, at least 70 years of age, at least 75 years of age, and including at least 80 years of age or greater. The term “elderly” also includes the groups of from 45 years of age to 100 years of age, and the group of from 55 years of age to 80 years of age.

The terms “administer,” “administering,” “administered,” or “administration” as used herein, unless otherwise specified, should be understood to include providing the nutritional composition to an individual, the act of consuming the nutritional composition, and combinations thereof. In addition, it should be understood that the methods disclosed herein (e.g., administering) may be practiced with or without doctor supervision or other medical direction.

The term “therapeutically effective amount” as used herein, unless otherwise specified, refers to a sufficient amount of a combination of active ingredients (e.g., EGCg and HMB) to decrease the level of intramuscular FGF2, and to exhibit a therapeutic effect. Exemplary therapeutic effects include one or more of: enhancing the regenerative capacity of muscle, maintaining muscle function, and improving muscle function. The exact amount required to be effective will vary from individual to individual, depending on the species, age, weight, lifestyle and general condition of the particular individual.

The term “nutritional liquid” as used herein, unless otherwise specified, refers to nutritional compositions in ready-to-drink liquid form, concentrated liquid form, and nutritional liquids made by reconstituting nutritional powders described herein prior to use. The nutritional liquid may also be formulated as a suspension, an emulsion, a solution, and so forth.

The terms “nutritional powder” or “reconstitutable powder” as used herein, unless otherwise specified, refer to nutritional compositions in flowable or scoopable form that can be reconstituted with water or another aqueous liquid prior to consumption and includes both spray dried and drymixed/dryblended powders.

The term “nutritional semi-solid” as used herein, unless otherwise specified, refers to nutritional compositions that are intermediate in properties, such as rigidity, between solids and liquids. Some semi-solid examples include puddings, yogurts, gels, gelatins, and doughs.

The term “nutritional semi-liquid” as used herein, unless otherwise specified, refers to nutritional compositions that are intermediate in properties, such as flow properties, between liquids and solids. Some semi-liquid examples include thick shakes, liquid yogurts, and liquid gels.

The term “serving” as used herein, unless otherwise specified, is intended to be construed as any amount which is intended to be consumed by an individual in one sitting or within one hour or less.

The term “muscle” as used herein, unless otherwise specified, refers to skeletal muscles as well as other non-skeletal, striated muscles such as diaphragm, extraocular muscle, and so forth.

The term “intramuscular” as used herein, unless otherwise specified, refers to all cellular parts that comprise a skeletal muscle group, including but not limited to myofibers, myoblasts, satellite cells, neurons, endothelial cells, pericytes, monocytes, macrophages, adipocytes, and fibroblasts.

The term “muscle mass” as used herein, unless otherwise specified, refers to the amount or size of muscle or muscle groups, as expressed by muscle weight, mass, area, or volume. Muscle mass may also be expressed as total lean body mass, lean body mass of a body compartment such as the leg, or cross-sectional area of a leg or arm compartment. The volume or mass of the muscle can be determined using any known or otherwise effective technique that provides muscle area, volume or mass, such as dual energy X-ray absoptiometry (DEXA), or using visual or imaging techniques such as magnetic resonance imaging (MRI) or computed tomography (CT) scans.

The term “muscle strength” as used herein, unless otherwise specified, refers to the amount of force a muscle, or muscle groups in sum, can exert. Muscle strength may be evaluated by a variety of methods such as grip strength, one repetition maximum strength test, time-dependent tests of muscle endurance, time-dependent tests of muscle fatigue, or time-dependent tests of muscle endurance and fatigue, and so forth.

The term “muscle function” as used herein, unless otherwise specified, refers to at least one of muscle mass and muscle strength.

The term “regeneration” as used herein, unless otherwise specified, refers to one of satellite cell-mediated, syncitial myofiber growth or myoblast-dependent de novo myofiber differentiation, whereby myofiber growth may also be myoblast-dependent and de novo myofiber differentiation may be satellite cell-dependent, and whereby the aforementioned regeneration increases muscle mass, decreases the amount of muscle atrophy, or both.

In a first exemplary embodiment, a nutritional composition for enhancing the regenerative capacity of muscle in an individual is provided. The nutritional composition comprises a source of EGCg in an amount sufficient to provide 0.1 to 1 gram of EGCg per serving of nutritional composition; and a source of beta-hydroxy-beta-methylbutyrate (HMB) in an amount sufficient to provide 0.5 to 3 grams of HMB per serving of the nutritional composition.

In a second exemplary embodiment, a method for enhancing the regenerative capacity of muscle in an elderly individual is provided. The method comprises administering to an elderly individual, a nutritional composition comprising a therapeutically effective amount of epigallocatechin-gallate (EGCg), and a therapeutically effective amount of beta-hydroxy-beta-methylbutyrate (HMB). Administration of the nutritional composition results in an enhancement of regenerative capacity of the elderly individual's muscle(s).

In a third exemplary embodiment, a method for reducing the intramuscular level of fibroblast growth factor (FGF2) of an elderly individual is provided. The method comprises administering a nutritional composition comprising a therapeutically effective amount of epigallocatechin-gallate (EGCg) and a therapeutically effective amount of beta-hydroxy-beta-methylbutyrate (HMB) to an elderly individual, wherein the HMB and EGCg are present in the composition in a weight ratio between 1:2 to 30:1. Administration results in a decrease in the intramuscular level of FGF2 in the muscle(s) of the elderly individual.

As previously mentioned, decreases in muscle function (including loss of muscle mass) may occur from a number of factors and conditions including, age, disuse (including recovery from inactivity), as well as muscle wasting diseases, such as, cachexia due to cancer, end stage renal disease (ESRD), acquired immune deficiency syndrome (AIDS), or chronic obstructive pulmonary disease (COPD). (For a discussion of impairment of muscle recovery in aged muscle, see generally: Hvid et al., J. Appl. Physiol. (2010) 109, 1628-1634; Suetta et al., J. Appl. Physiol. (2009) 107, 1172-1180; and Magne et al., J Physiol 589.3 (2011) pp 511-524; For a discussion of impairment of muscle recovery due to disuse, see generally: Hao et al., Am. J. Physiol. Regul. Integr. Comp. Physiol. (2011) 301, R701-R715; Mozdziak et al., J. Appl. Physiol. (2001) 91, 183-190.)

As mentioned above, the exemplary nutritional compositions and methods disclosed herein include an amount of, or the administration of an amount of, a combination of EGCg and HMB effective to decrease the level of intramuscular FGF2. Fibroblast growth factor 2 (i.e., FGF2, bfgf, FGFB, FGF-2, heparin-binding growth factor 2, HBGF-2, prostatropin) is a secreted growth factor involved in cell proliferation and differentiation, tissue development and angiogenesis, and neurotrophic processes.

A mature adult contracting muscle cell, or is a multi-nucleated, syncitial elongated, cylinder-like cell (i.e., myofiber), generated by the prior and ongoing fusions of individual mono-nucleated cells (e.g., myoblasts and satellite cells) that populate muscle tissue. During embryonic development, this process of multiple cell fusions is called myogenesis. In adults, this process, mediated in part by myoblasts and satellite cells, is part of the normal repair, maintenance, renewal, and hypertrophic response of muscle tissue. Perpetual regeneration and hypertrophy, though, is negatively regulated to prevent aberrant over-amplification or growth of muscle tissue. This negative regulation increases with age, as the number of functional satellite cells decreases with age. Consequently, the regenerative capacity of muscle is decreased with age.

The role of FGF2 signaling in skeletal muscle is, as yet, not fully understood, although, several studies have shown its role in other processes. For example, Ortega et al. showed that FGF2 deficient phenotypes in genetic knockout mice are viable, fertile and indistinguishable from littermates by visual inspection, however, neuronal density was decreased in the frontal motor cortex and excisional wound healing was delayed. Ortega et al., Proc. Natl. Acad. Sci. U.S.A., (1998) 95, 5672-5677. FGF2-deficient mice also showed decreased vascular smooth muscle contractility, low blood pressure and thrombocytosis, but normal proliferative response to mechanical vessel injury. Zhou et al., Nat. Med., (1998) 4, 201-207. Further, in FGF2-deficient mice, it was shown that FGF2 is required for differentiation of cardiogenic precursors, yet FGF2 did not regulate precursor cell number. Rosenblatt-Velin et al., J. Clin. Invest., (2005) 115, 1724-1733. More recently, it has been demonstrated, in mice, that FGF2 protein signaling defines the self-renewing capacity of muscle stem cells. This signaling occurs along the length of mature contracting myofibers where satellite cells directly interact with the myofiber, and whose interaction is critical for satellite cell quiescence, i.e., not proliferative. Chakkalakal et al., Nature, (2012) 490, 355-360. This quiescence is then, in turn, necessary for stem cell self-renewal and regenerative capacity. Continued satellite cell cycling (i.e., non-quiescence) decreases the ability of daughter cells to self-renew, favoring limited differentiation and apoptosis. Further, FGF2 was found to localize in the basal lamina of myofibers where satellite cells and myofibers interact. This FGF2 expression increases with age and induces cycling (non-quiescence) of satellite cells.

The general inventive concepts recognize that inhibiting or decreasing the level of intramuscular FGF2 in aged muscle promotes muscle regeneration and impedes muscle loss. Improved muscle regeneration and hypertrophy can lead to increases in muscle mass and muscle strength, and thereby decrease muscle function decline, improve muscle function, or both. Unexpectedly, a combination of EGCg and HMB were found to reduce the level of intramuscular FGF2. Therefore, administration of a combination of EGCg and HMB is expected to lead to improved muscle regeneration and hypertrophy, as well as increases in muscle mass and muscle strength, or combinations thereof.

As used herein, the term “EGCg” refers to epigallocatechin-3-gallate, or a source thereof. Similarly, as used herein, the term “HMB” refers to beta-hydroxy-beta-methylbutyrate, or a source thereof. As previously mentioned, it has been discovered that administration of a combination of a therapeutically effective amount of EGCg (or source thereof) and a therapeutically effective amount of HMB (or a source thereof) decreases the level of intramuscular FGF2, which in turn slows age-related declines in muscle function, enhances the regenerative capacity of muscle, or both.

In accordance with the exemplary nutritional compositions and methods disclosed herein, the EGCg and HMB can be formulated in a suitable composition (e.g., a nutritional composition) and then administered to an individual in a form adapted to the chosen route or course of administration. The compositions disclosed herein, and utilized in the disclosed methods, include those suitable for oral administration. Oral administration, as defined herein, includes any form of administration in which the active ingredients (EGCg and HMB) pass through the esophagus of the individual. For example, oral administration includes nasogastric intubation, in which a tube is run from through the nose to the stomach of the individual to administer food or drugs.

In certain exemplary embodiments, and optionally according to the first exemplary embodiment, the EGCg and HMB are administered to the individual orally. Generally, the combination of a therapeutically effective amount of EGCg and a therapeutically effective amount of HMB may be provided in any form suitable for oral consumption by the individual. For example, the combination may be provided as caplets, tablets, pills, capsules, chewable tablets, quick dissolve tablets, effervescent tablets, solutions, suspensions, emulsions, multi-layer tablets, bi-layer tablets, soft gelatin capsules, hard gelatin capsules, lozenges, chewable lozenges, beads, granules, particles, microparticles, dispersible granules, cachets, and combinations thereof.

The EGCg used in the nutritional compositions and administered according to the methods disclosed herein may be provided by natural or synthetic sources. Suitable sources of EGCg for use in the methods disclosed herein are green tea-based sources including, but not limited to, green tea extracts in which EGCg alone, or in combination with other polyphenol compounds (e.g., flavan-3-ols), are isolated from green tea as an extract. Examples of such suitable green tea extracts are in the form of a liquid with a high concentration of the polyphenols, a solid (e.g., a powder), and mixtures thereof. In certain embodiments where a green tea extract is utilized, the extract is decaffeinated such that it contains less than 1% by weight caffeine, or even less than 0.5% by weight caffeine. In addition to containing EGCg, suitable green tea extracts used in the nutritional compositions and administered according to the methods disclosed herein may contain other polyphenols including other flavan-3-ols such as catechin (e.g., (+)-catechin, also known as “C”), epicatechin (“EC”), gallocatechin (“GC”), epigallocatechin (“EGC”), and epicatechin gallate (“ECg”), and stereoisomers thereof flavones such as apigenin, isoviloxin, sapotarin, and vicenin-2; flavonols such as kaempherol, quercetin, and myricetin; condensed flavanoids; and tannin glycosides. Accordingly, in certain exemplary embodiments, in addition to EGCg, the individual is administered, and in certain exemplary embodiments according to the first embodiment, the nutritional composition comprises one or more flavan-3-ols selected from the group consisting of C, EC, GC, EGC, and ECg.

In certain exemplary embodiments, sources of EGCg other than green tea-based sources may be utilized. These sources include, but are not limited to, oolong tea-based sources such as oolong tea, oolong tea extracts, and the like; white tea-based sources such as white tea, white tea extracts, and the like; macha tea, macha tea extracts, and the like; yellow tea, yellow tea extracts, and the like; and dark tea (i.e., Chinese dark tea), dark tea extracts, and the like.

In certain exemplary embodiments, when the EGCg is provided in the nutritional composition as part of a green tea extract, the green tea extract contains at least 10% by weight EGCg. In exemplary embodiments, when the EGCg is provided as part of a green tea extract, the green tea extract contains at least 25% by weight EGCg. In exemplary embodiments, the EGCg is provided as part of a green tea extract that contains 10-100% by weight EGCg. In exemplary embodiments, the EGCg is provided as part of a green tea extract that contains 25-100% by weight EGCg. In exemplary embodiments, the EGCg is provided as part of a green tea extract that contains 50-100% by weight EGCg. In exemplary embodiments, the EGCg is provided as part of a green tea extract that contains 60-100% by weight EGCg. In exemplary embodiments, the EGCg is provided as part of a green tea extract that contains 70-100% by weight EGCg. In exemplary embodiments, the EGCg is provided as part of a green tea extract that contains 80-100% by weight EGCg, including 90-100% by weight EGCg.

In certain exemplary embodiments, the nutritional composition comprises 0.1 to 1 grams of EGCg per serving. Further, in other exemplary embodiments, the nutritional composition comprises 0.25 grams to 1 grams of EGCg per serving. In other exemplary embodiments, the nutritional composition comprises 0.5 grams to 0.75 grams of EGCg per serving. In other exemplary embodiments, the nutritional composition comprises 0.25 gram to 0.5 grams of EGCg per serving. Examples of commercially available sources of EGCg provided as part of a green tea extract include Teavigo® (>90% EGCg) (DSM, Netherlands) and SUNPHENON® 90D (Taiyo International, Inc., Minneapolis, Minn.).

In addition to EGCg, the nutritional compositions also comprise HMB (or a source thereof). HMB is a metabolite of the essential amino acid leucine. As used herein, the terms HMB and β-hydroxy-β-methylbutyrate should be understood to encompass its multiple forms including, but not limited to, salts (including anhydrous salts), the free acid, esters, and lactones, unless it is clear from the context that only one form is meant. One suitable form of HMB that may be utilized is the calcium salt of HMB, also designated as Ca-HMB, which is most typically the monohydrate calcium salt. The HMB used can come from any source. Calcium HMB monohydrate is commercially available from Technical Sourcing International (TSI) of Salt Lake City, Utah. When referring to amounts of HMB herein, the amounts are based on the assumption that the HMB is being provided as Ca-HMB, unless specifically indicated otherwise. Non-limiting examples of suitable salts of HMB (hydrated or anhydrous) for use herein include sodium, potassium, chromium, calcium, and other non-toxic salt forms. In certain exemplary embodiments, the term HMB may refer to other metabolites of leucine, such as, alpha-keto-isocaproate, alpha-hydroxyisocaproic acid, or combinations thereof (including combinations with beta-hydroxy-beta-methylbutyrate).

In certain exemplary embodiments, the nutritional composition comprises 0.5 to 3 grams of HMB per serving. Further, in other exemplary embodiments, the nutritional composition comprises 0.5 grams to 2.5 grams of HMB per serving of the nutritional composition. In other exemplary embodiments, the nutritional composition comprises 0.5 grams to 2 grams of HMB per serving of the nutritional composition. In other exemplary embodiments, the nutritional composition comprises 1 gram to 2 grams of HMB per serving of the nutritional composition. In other exemplary embodiments, the nutritional composition comprises 1 gram to 1.5 grams of HMB per serving of the nutritional composition.

In accordance with certain exemplary methods disclosed herein, compositions including a therapeutically effective amount of EGCg in combination with a therapeutically effective amount of HMB, can be provided to an individual in need thereof in one or more doses, or servings, over a period of time. In certain exemplary embodiments according to the methods disclosed herein, a therapeutically effective amount of EGCg in combination with a therapeutically effective amount of HMB, is provided or administered to an individual in need thereof in two servings per day. In other exemplary embodiments, a therapeutically effective amount of EGCg in combination with a therapeutically effective amount of HMB is administered to an individual in need thereof in two servings, three servings, or four servings or more per day. In certain exemplary embodiments, EGCg is administered in an amount of 0.5 to 1.5 grams per day, and HMB is administered in an amount of 2 to 4 grams per day.

As previously mentioned, the level of intramuscular FGF2 is decreased in an individual by administration of a therapeutically effective amount of EGCg in combination with a therapeutically effective amount of HMB. In certain exemplary embodiments, the amount of EGCg administered to the individual is different than the amount of HMB administered to the individual in order to achieve the desired effect. In such exemplary embodiments, the amounts of EGCg and HMB may be expressed as a ratio (wt/wt). Accordingly, in certain exemplary embodiments, the HMB and EGCg are present in the nutritional composition in a ratio of 1:2 to 30:1 (wt HMB/wt EGCg). In certain other exemplary embodiments the HMB and EGCg are present in the nutritional composition in a ratio of 3:1 to 10:1 (wt HMB/wt EGCg).

Certain exemplary embodiments include administering to an individual in need thereof an amount of EGCg in combination with an amount of HMB effective to decrease the level of intramuscular FGF2, to enhance the regenerative capacity of muscle, or both. The decrease in intramuscular FGF2 also decreases FGF2-associated signaling in the muscle, which may promote satellite cell quiescence. These results, in turn, lead to positive therapeutic effects in the muscle of the individual in need thereof, such as maintenance of muscle function, increased muscle endurance, and increased muscle hypertrophy.

In certain exemplary embodiments, the individual in need thereof is a human. In certain exemplary embodiments, the individual in need thereof is an elderly human. In certain exemplary embodiments, the individual in need thereof is an individual who is experiencing muscle function decline. In certain exemplary embodiments, the individual in need thereof is an individual in need of enhancement of muscle regenerative capacity by virtue of having one or more of sarcopenia, cachexia, chronic obstructive pulmonary disease (COPD), end stage renal disease (ESRD), acquired immune deficiency syndrome (AIDS); an individual who is bedridden or otherwise immobile and suffers from muscle disuse; or combinations thereof. Symptoms of decreasing muscle regenerative capacity include, but are not limited to, decreased muscle growth, decreased muscle oxygenation, muscle inflammation, and increased muscle catabolism. Such symptoms may manifest as a result of aging, sarcopenia, cachexia, inactivity, immobility (e.g., bed rest or due to a cast, etc.), AIDS, ESRD, COPD, or combinations thereof. In certain other exemplary embodiments, the individual in need thereof is hospitalized. In yet other exemplary embodiments, the individual in need thereof is undergoing rehabilitation subsequent to a period of injury, disease, surgery, immobilization, hospitalization, or combinations thereof.

As used herein, the phrase “enhancing the regenerative capacity of muscle” should be understood to include one or more of reducing the rate of muscle function decline (including age-related decline in muscle function), maintaining muscle function, or improving muscle function. As noted above, muscle function includes at least one of muscle mass and muscle strength. In addition, in certain exemplary embodiments, “enhancing the regenerative capacity of muscle” should be understood to include one or more of increasing muscle growth, increasing muscle endurance, reducing muscle inflammation, decreasing muscle catabolism, increasing muscle mass, and increasing muscle strength.

In certain exemplary embodiments, the regenerative capacity of muscle (evidenced by changes in muscle function) in an individual may be evaluated by a wide variety of methods. For example, muscle function, in terms of muscle mass, in an individual may be determined by using any known or otherwise effective technique that provides muscle area, volume, or mass, such as DEXA, or using visual or imaging techniques such as MRI or CT scans. Further, muscle function in terms of muscle mass may be determined through electrical impedence methods, such as, bioelectrical impedance analysis (BIA) and bioelectrical impedance spectrometry (BIS). In addition, muscle function in an individual, in terms of muscle strength, can be quantitatively measured using acute tests of maximum force, time-dependent tests of muscle endurance, time dependent tests of muscle fatigue, time dependent tests of muscle endurance and fatigue, or combinations thereof. Furthermore, muscle function in an individual may be measured by using a grip meter, by evaluating lower extremity strength using equipment to measure isokinetic knee extensor or knee flexor strength, and by measuring gait and balance (e.g., Tinetti Gait and Balance test). Moreover, muscle regeneration in an individual may be measured by obtaining muscle tissue samples (e.g., needle biopsy) and performing assays (e.g., ELISA, western blot, quantitative reverse transcription-polymerase chain reaction, RNase protection assay) to measure intramuscular levels of FGF2 protein or messenger ribonucleic acid. Furthermore, in certain exemplary embodiments, muscle regeneration in an individual may be measured by obtaining a bodily sample, other than muscle tissue, to serve as a proxy for a muscle tissue sample, such as, for example: a blood, urine, saliva or other fluid sample; and performing assays (e.g., ELISA, western blot, quantitative reverse transcription-polymerase chain reaction, RNase protection assay) to measure intramuscular levels of FGF2 protein or messenger ribonucleic acid.

In certain exemplary embodiments, “enhancing the regenerative capacity of muscle,” as used herein, also refers to the maintenance of muscle function in the individual. In this context, maintenance of muscle function in the individual refers to retaining an amount of muscle function that corresponds to a measurement of the muscle function of the individual prior to initiating the methods disclosed herein, or a percentage thereof. Accordingly, in various exemplary embodiments of the methods disclosed herein, administering an amount of a combination of EGCg and HMB effective to decrease the level of intramuscular FGF2 results in maintaining 100% of the muscle function of the individual, or in other embodiments lesser amounts. For example, in certain exemplary embodiments, the methods result in maintaining at least 50% muscle function. In certain other exemplary embodiments, the methods result in maintaining at least 60% muscle function. In other certain exemplary embodiments, the methods result in maintaining at least 70% muscle function. In certain other exemplary embodiments, the methods result in maintaining at least 80% muscle function. In certain other exemplary embodiments, the methods result in maintaining at least 90% muscle function. In certain other exemplary embodiments, the methods result in maintaining at least 95% muscle function. In certain other exemplary embodiments, the methods result in maintaining muscle function in any amounts ranging from 50% to 100%, including 50% to 80%, 50% to 90%, 60% to 80%, and 60% to 90%. In certain other exemplary embodiments muscle function decline is entirely prevented; in other words, the individual maintains 100% muscle function, or even increases muscle function. Generally, when muscle function in an individual is “maintained” by more than 100%, this result is described herein as an improvement in muscle function.

Certain exemplary embodiments disclosed herein result in an improvement of muscle function in an individual. The terms “improve,” “improves,” “improvement,” or “improving” when used in connection with muscle function refers to an increase in muscle function, or alternatively, maintenance of muscle function above 100% as compared to a period of time before application of the compositions or methods disclosed herein. For example, in an exemplary embodiment, administering to an individual an amount of a combination of EGCg and HMB effective to decrease the level of intramuscular FGF2 can increase the individual's muscle function by at least 10%, such as 10% to 100%. In certain exemplary embodiments, muscle function can be improved by at least 1% (i.e., 1 to 100%). In certain other exemplary embodiments, muscle function can be improved by at least 5%. In certain other exemplary embodiments, muscle function can be improved by at least 20%. In certain other exemplary embodiments, muscle function can be improved by at least 30%. In certain other exemplary embodiments, muscle function can be improved by at least 40%. In certain other exemplary embodiments, muscle function can be improved by at least 50%. In certain other exemplary embodiments, muscle function can be improved by at least 60%. In certain other exemplary embodiments, muscle function can be improved by at least 70%. In certain other exemplary embodiments, muscle function can be improved by at least 80%. In certain other exemplary embodiments, muscle function can be improved by 90%, or more.

When measuring changes in muscle function including: an improvement in muscle function, a reduction in muscle function decline, or maintenance of muscle function, a first measurement of the muscle function of the individual is performed prior to initiating the methods disclosed herein. In certain exemplary embodiments of the methods disclosed herein, the first measurement is performed a week (e.g., 1-7 days or 7 days) before initiation of the methods disclosed herein. Next, a second measurement of the muscle function of the individual is performed at some time point after initiating the methods disclosed herein, and the second measurement is compared to the first measurement. Notably, the comparison of the second measurement to the first measurement may not show immediate results using the aforementioned measurement techniques. The resulting effect may take days, weeks, or months of regular administration of a combination of a therapeutically effective amount of EGCg and a therapeutically effective amount of HMB (or nutritional compositions containing the EGCg and HMB) according to the dosages and in the intervals previously described herein to obtain the stated measurable muscle function results described above.

In certain exemplary embodiments, the level of intramuscular FGF2 is decreased (or reduced) by administration of the EGCg and HMB containing compositions. In certain exemplary embodiments the level of intramuscular FGF2 is reduced by 1 to 50%. In certain other exemplary embodiments the level of intramuscular FGF2 is reduced by 1 to 40%. In certain other exemplary embodiments the level of intramuscular FGF2 is reduced by 1 to 30%. In certain other exemplary embodiments the level of intramuscular FGF2 is reduced by 1 to 20%. In certain other exemplary embodiments the level of intramuscular FGF2 is reduced by 1 to 10%. In certain other exemplary embodiments the level of intramuscular FGF2 is reduced by 10 to 50%. In certain other exemplary embodiments the level of intramuscular FGF2 is reduced by 20 to 50%. In certain other exemplary embodiments the level of intramuscular FGF2 is reduced by 20 to 40%. In certain other exemplary embodiments the level of intramuscular FGF2 is reduced by 30 to 40%. Similarly to that discussed previously, when measuring a decrease in intramuscular FGF2 level, a first measurement of the intramuscular FGF2 level of the individual is performed prior to initiating the methods disclosed herein. In certain embodiments of the methods disclosed herein, the first measurement is performed a week (e.g., 1-7 days or 7 days) before initiation of the methods disclosed herein. Next, a second measurement of the intramuscular FGF2 level of the individual is performed at some time point after initiating the methods disclosed herein, and the second measurement is compared to the first measurement. Notably, the comparison of the second measurement to the first measurement may not show immediate results using the aforementioned measurement techniques. The resulting effect may take days, weeks, or months of regular administration of a combination of a therapeutically effective amount of EGCg and a therapeutically effective amount of HMB (or nutritional compositions containing the EGCg and HMB) according to the dosages and in the intervals previously described herein to obtain the stated measurable muscle function results described above. Alternatively, the intramuscular FGF2 level of the individual is compared to an average of several (e.g., 3-5) measurements of intramuscular FGF2 levels from the individual performed prior to initiating the methods disclosed herein. Additionally, the intramuscular FGF2 level of the individual, after initiating the methods disclosed herein, may be compared to a control level of intramuscular FGF2 (i.e., an average concentration) of samples from individuals who have yet to initiate the methods disclosed herein.

As discussed above, an enhancement in the muscle regenerative capacity, an improvement in muscle function, or a decrease in the level of intramuscular FGF2 in an individual may be measured in a variety of ways including, for example, obtaining muscle tissue samples (e.g., needle biopsy) from the individual prior to initiating the methods disclosed herein and at a time point after initiating the methods disclosed herein, and performing standard assays (e.g., e.g., ELISA, western blot, quantitative reverse transcription-polymerase chain reaction, RNase protection assay) to measure and compare intramuscular levels of FGF2. In addition, an animal study (e.g., according to Example 3 or a similar study) may be used to show that administration of a combination of EGCg and HMB (or a nutritional composition containing EGCg and HMB) according to the methods disclosed herein results in a decrease in muscle function decline or an improvement in muscle function.

In accordance with the various exemplary embodiments disclosed herein, a therapeutically effective amount of EGCg (or a source thereof) in combination with a therapeutically effective amount of HMB (or a source thereof) can be administered to (or consumed by) an individual in need thereof one or more times per day for a period suitable to achieve the desired effect. For example, a composition comprising both EGCg and HMB can be administered to an individual in need thereof every day for at least a week. In certain exemplary embodiments the composition comprising both EGCg and HMB is administered every day for at least two weeks. In certain other exemplary embodiments the composition comprising both EGCg and HMB is administered every day for at least a month. In certain other exemplary embodiments the composition comprising both EGCg and HMB is administered every day for at least 6 months. In certain other exemplary embodiments the composition comprising both EGCg and HMB is administered every day for a year or more. As another example, a composition comprising both EGCg and HMB can be administered to an individual in need thereof twice a day for at least a week. In certain other exemplary embodiments the composition comprising both EGCg and HMB is administered twice a day for at least two weeks. In certain other exemplary embodiments the composition comprising both EGCg and HMB is administered twice a day for at least a month. In certain other exemplary embodiments the composition comprising both EGCg and HMB is administered twice a day for at least 6 months. In certain other exemplary embodiments the composition comprising both EGCg and HMB is administered twice a day for a year or more. Within the context of administering a dose to an individual, every day is intended to reflect a period of time in which an individual has been instructed to receive the combination of EGCg and HMB every day, and in which the combination of EGCg and HMB is actually administered to the individual for at least 70% of the days during the desired period of administration.

In certain exemplary embodiments, the therapeutically effective amount of EGCg in combination with the therapeutically effective amount of HMB (or a composition containing both EGCg and HMB) is chronically administered. “Chronically administering” refers, in one exemplary embodiment, to regular administration which is provided indefinitely. In other exemplary embodiments, the term refers to regular administration for a significant period of time. For example, in certain exemplary embodiments chronic administration can include regular administration for at least one month. In certain other exemplary embodiments chronic administration can include regular administration for at least 6 weeks. In certain other exemplary embodiments chronic administration can include regular administration for at least two months. In certain other exemplary embodiments chronic administration can include regular administration for at least 3 months. In certain other exemplary embodiments chronic administration can include regular administration for at least 4 months. In certain other exemplary embodiments chronic administration can include regular administration for at least 5 months. In certain other exemplary embodiments chronic administration can include regular administration for at least 6 months. In certain other exemplary embodiments chronic administration can include regular administration for at least 9 months. In further exemplary embodiments, chronic administration refers to regular administration for at least 1 year. In certain other exemplary embodiments chronic administration can include regular administration for at least 1.5 years. In certain other exemplary embodiments chronic administration can include regular administration for at least 2 years. In certain other exemplary embodiments chronic administration can include regular administration for more than 2 years. “Regular administration” refers to administration according to a schedule where it is intended that the individual in need thereof will receive the combination of EGCg and HMB at regular intervals.

As used herein, “regular intervals” refers to administration in a repeating, periodic fashion where the time between administrations is approximately (or intended to be approximately) the same. In certain exemplary embodiments, administration at regular intervals includes daily administration or weekly administration. In certain exemplary embodiments, the term refers to administration 1-2 times per week. In certain exemplary embodiments, the term refers to administration 1-3 times per week. In certain exemplary embodiments, the term refers to administration 2-3 times per week. In certain exemplary embodiments, the term refers to administration 1-4 times per week. In certain exemplary embodiments, the term refers to administration 1-5 times per week. In certain exemplary embodiments, the term refers to administration 2-5 times per week. In certain exemplary embodiments, the term refers to administration 3-5 times per week. In certain exemplary embodiments, the term refers to administration 1-2 times per day. In certain exemplary embodiments, the term refers to administration 1-3 times per day. In certain exemplary embodiments, the term refers to administration 1-4 times per day. In certain exemplary embodiments, the term refers to administration 2-3 times per day. In certain exemplary embodiments, the term refers to administration 2-4 times per day. In certain exemplary embodiments, the term refers to administration 3-4 times per day. In certain exemplary embodiments, the term refers to administration 2-5 times per day. In certain exemplary embodiments, the term refers to administration 3-5 times per day. In certain exemplary embodiments, the term refers to administration 4-5 times per day.

As discussed herein, a therapeutically effective amount of the combination of EGCg and HMB refers to a sufficient amount of EGCg and HMB in combination to decrease the level of intramuscular FGF2 and to exhibit a resultant therapeutic effect (e.g., maintenance of muscle function, enhancement of muscle regenerative capacity). The exact amount of the combination of EGCg and HMB required to achieve the decrease in intramuscular FGF2 and therapeutic effect may be varied to suit a particular individual or class of similarly suited individuals. Similarly, the form of delivery of the combination EGCg and HMB (i.e., the form of the EGCg and HMB containing nutritional composition) may be varied to suit a particular individual or class of similarly suited individuals.

As previously discussed, in certain exemplary embodiments, the EGCg and HMB are provided as part of a nutritional composition. In certain exemplary embodiments, the nutritional compositions are formulated as, and intended for consumption in, any known or otherwise suitable oral composition form. Any solid, liquid, semi-solid, semi-liquid, or powder composition form, including combinations or variations thereof, are suitable for use herein, provided that such forms allow for safe and effective oral delivery to the individual via oral consumption of the ingredients as also defined herein.

In certain exemplary embodiments, the nutritional composition is a solid nutritional composition. Non-limiting examples of solid nutritional compositions include snack and meal replacement compositions, including those formulated as bars, sticks, cookies or breads or cakes or other baked goods, frozen liquids, candy, breakfast cereals, powders or granulated solids or other particulates, snack chips or bites, frozen or retorted entrees and so forth. In certain exemplary embodiments, when the nutritional composition is a solid composition, the serving is within a range of 25 grams to 150 grams.

In certain exemplary embodiments, the nutritional composition may be a nutritional liquid. Non-limiting examples of nutritional liquids include snack and meal replacement compositions, hot or cold beverages, carbonated or non-carbonated beverages, juices or other acidified beverages, milk or soy-based beverages, shakes, coffees, teas, compositions for administration by nasogastric intubation, and so forth. Generally, the nutritional liquids are formulated as suspensions or emulsions, but the nutritional liquids can also be formulated in any other suitable forms such as clear liquids, solutions, liquid gels, liquid yogurts, and so forth.

In certain exemplary embodiments, where the nutritional composition is a liquid, the serving is within a range of 30 milliliters to 500 milliliters (˜1 fl. oz. to ˜17 fl. oz.). In certain other exemplary embodiments, where the nutritional composition is a liquid, the serving is 237 milliliters (˜8 fl. oz.). In certain other exemplary embodiments, where the nutritional composition is a liquid, the serving is 125 milliliters (˜4 fl. oz.). In certain other exemplary embodiments, where the nutritional composition is a liquid, the serving is 177 milliliters to 417 milliliters (˜6 fl. oz. to ˜14 fl. oz.). In yet other exemplary embodiments, where the nutritional composition is a liquid, the serving is 207 milliliters to 266 milliliters (˜7 fl. oz. to ˜9 fl. oz.). In still other exemplary embodiments, where the nutritional composition is a liquid, the serving is 30 milliliters to 75 milliliters (˜1 fl. oz. to ˜2.5 fl. oz.). In certain exemplary embodiments, where the nutritional composition is administered as a liquid, one serving to 14 servings of the nutritional composition is administered to the individual per week.

In certain other exemplary embodiments, the nutritional composition may be formulated as semi-solid or semi-liquid compositions (e.g., puddings, gels, yogurts, etc.), as well as more traditional forms such as capsules, tablets, caplets, pills, and so forth.

The nutritional compositions according to exemplary embodiments disclosed herein are useful to provide sole, primary, or supplemental sources of nutrition, as well as providing one or more of the benefits as described herein. Accordingly, in certain exemplary embodiments, the nutritional compositions disclosed herein may include one or more macronutrients. For example, in certain embodiments, the nutritional composition comprises at least one source of fat, at least one source of carbohydrates, at least one source of protein, or combinations thereof. In certain other exemplary embodiments, the nutritional composition comprises at least one source of protein, at least one source of carbohydrates, but no source of fat. In certain exemplary embodiments, the nutritional composition provides up to 1000 kcal of energy per serving or dose. In certain other exemplary embodiments, the nutritional composition provides 20 kcal to 900 kcal of energy per serving. In certain other exemplary embodiments, the nutritional composition provides 25 kcal to 700 kcal of energy per serving. In certain other exemplary embodiments, the nutritional composition provides 50 kcal to 500 kcal of energy per serving. In certain other exemplary embodiments, the nutritional composition provides 100 kcal to 450 kcal of energy per serving. In certain other exemplary embodiments, the nutritional composition provides 150 kcal to 400 kcal of energy per serving.

In certain exemplary embodiments, the nutritional composition comprises at least one source of protein in an amount sufficient to provide 5 to 50 grams of protein per serving of the nutritional composition. In certain other exemplary embodiments, the nutritional compositions comprise 5 to 40 grams of protein per serving of the nutritional composition. In certain other exemplary embodiments, the nutritional compositions comprise 10 to 30 grams of protein per serving of the nutritional composition. In certain other exemplary embodiments, the nutritional compositions comprise 10 to 25 grams of protein per serving of the nutritional composition. In certain other exemplary embodiments, the nutritional compositions comprise 5 to 20 grams of protein per serving of the nutritional composition.

Virtually any source of protein may be used so long as it is suitable for use in oral nutritional compositions and is otherwise compatible with any other selected ingredients or features in the nutritional composition. The source of protein may include, but is not limited to, intact, hydrolyzed, and partially hydrolyzed protein, which may be derived from any known or otherwise suitable source such as milk (e.g., casein, whey), animal (e.g., meat, fish, earthworm), cereal (e.g., rice, corn), vegetable (e.g., soy, pea), insect (e.g., cricket, locust), and combinations thereof. Non-limiting examples of the source of protein include whey protein concentrates, whey protein isolates, whey protein hydrolysates, acid caseins, sodium caseinates, calcium caseinates, potassium caseinates, casein hydrolysates, milk protein concentrates, milk protein isolates, milk protein hydrolysates, nonfat dry milk, condensed skim milk, soy protein concentrates, soy protein isolates, soy protein hydrolysates, pea protein concentrates, pea protein isolates, pea protein hydrolysates, collagen proteins, collagen protein concentrates, collagen protein isolates, insect protein isolates, and combinations thereof. In addition, the at least one source of protein in an amount sufficient to provide 5 to 50 grams of protein per serving may comprise any one source of protein or any combination of any of the various sources of protein provided in the non-limiting list presented above.

In addition to the at least one source of protein, in certain exemplary embodiments, the nutritional composition further comprises at least one source of carbohydrates, at least one source of fat, or combinations thereof. Therefore, in certain exemplary embodiments the nutritional composition further comprises at least one source of carbohydrates, while in other exemplary embodiments the nutritional composition further comprises at least one source of fat, and yet in other exemplary embodiments the nutritional composition further comprises at least one source of carbohydrates and at least one source of fat.

In certain exemplary embodiments wherein the nutritional composition comprises carbohydrates, 10 grams to 110 grams of at least one source of carbohydrates per serving are present in the nutritional composition. In other exemplary embodiments, the nutritional composition comprises 10 grams to 90 grams of at least one source of carbohydrates per serving. In other exemplary embodiments, the nutritional composition comprises 10 grams to 65 grams of at least one source of carbohydrates per serving. In other exemplary embodiments, the nutritional composition comprises 10 grams to 55 grams of at least one source of carbohydrates per serving. In other exemplary embodiments, the nutritional composition comprises 10 grams to 25 grams of at least one source of carbohydrates per serving.

The at least one source of carbohydrates suitable for use in certain exemplary embodiments of the nutritional compositions disclosed herein may be simple, complex, or variations or combinations thereof. Generally, any source of carbohydrates may be used so long as it is suitable for use in oral nutritional compositions and is otherwise compatible with any other selected ingredients or features present in the nutritional composition. Non-limiting examples of a source of carbohydrates suitable for use in the nutritional compositions described herein include maltodextrin, hydrolyzed or modified starch or cornstarch, glucose polymers, corn syrup, corn syrup solids, rice-derived carbohydrates, sucrose, glucose, fructose, lactose, high fructose corn syrup, honey, sugar alcohols (e.g., maltitol, erythritol, sorbitol, etc.), isomaltulose, sucromalt, pullulan, potato starch, and other slowly-digested carbohydrates, dietary fibers including, but not limited to, fructooligosaccharides (FOS), galactooligosaccharides (GOS), oat fiber, soy fiber, gum arabic, sodium carboxymethylcellulose, methylcellulose, guar gum, gellan gum, locust bean gum, konjac flour, hydroxypropyl methylcellulose, tragacanth gum, karaya gum, gum acacia, chitosan, arabinoglactins, glucomannan, xanthan gum, alginate, pectin, low and high methoxy pectin, cereal beta-glucans (i.e., oat beta-glucan, barley beta-glucan), carrageenan, psyllium, Fibersol™, other resistant starches, and combinations thereof.

As previously discussed, in certain exemplary embodiments, the nutritional composition further comprises at least one source of fat. In other exemplary embodiments, the nutritional composition comprises no fat, or essentially no fat (i.e., less than 0.5 grams of fat per serving). In certain exemplary embodiments where the nutritional composition contains fat, the nutritional composition comprises 0.5 grams to 45 grams of at least one source of fat per serving of the nutritional composition. In certain other exemplary embodiments, the nutritional composition comprises 5 grams to 25 grams of at least one source of fat per serving. In certain other exemplary embodiments, the nutritional composition comprises 10 grams to 20 grams of at least one source of fat per serving. In certain other exemplary embodiments, the nutritional composition comprises 10 grams to 15 grams of at least one source of fat per serving.

In general, any source of fat may be used so long as it is suitable for use in oral nutritional compositions and is otherwise compatible with any other selected ingredients or features present in the nutritional composition. The source of fat may be derived from plants, animals, or combinations thereof. Non-limiting examples of suitable sources of fat for use in the nutritional compositions described herein include coconut oil, fractionated coconut oil, soy oil, corn oil, olive oil, safflower oil, high oleic safflower oil, high gamma linoleic acid (GLA) safflower oil, MCT oil (medium chain triglycerides), sunflower oil, high oleic sunflower oil, palm and palm kernel oils, palm olein, canola oil, marine oils, cottonseed oils, eicosapentaenoic acid, docosahexaenoic acid, gamma-linolenic acid, conjugated linolenic acid from any source, or combinations thereof.

In certain exemplary embodiments, the nutritional composition further comprises one or more functional ingredients that increase muscle protein synthesis, or decrease muscle protein degradation, or reduce muscle necrosis or apoptosis, or combinations thereof. For example, in certain exemplary embodiments disclosed herein, the nutritional composition further comprises a functional ingredient selected from the group consisting of: a branched-chain amino acid selected from the group consisting of leucine, isoleucine, valine, metabolites of any of the foregoing branched-chain amino acids including alpha-ketoisocaproic acid and alpha-hydroxyisocaproic acid, alpha-ketoisovaleric acid, alpha-hydroxyisovaleric acid, beta-hydroxyisobutyric acid, 2-oxo-3-methylvaleric acid, 2-hydroxy-3-methylvaleric acid, 3-hydroxy-2-ethylpropionic acid, 3-hydroxy-2-methylbutyric acid, and combinations thereof; β-alanine; Vitamin D; creatine; carnitine; carnosine; anserine; taurine; α-hydroxyisovaleric acid; α-ketoglutarate, citrulline, arginine, or a combination thereof.

In accordance with certain exemplary embodiments, the nutritional composition is formulated as a liquid. In certain such exemplary embodiments, the nutritional composition is a clear liquid having a pH ranging from 2 to 5, and also having no more than 0.5% fat by weight of the nutritional composition. The limited amount of fat contributes to the desired clarity and is compatible with a pH of 2 to 5 for certain embodiments of the nutritional composition. Typically, liquid nutritional compositions desired to be clear, or at least substantially translucent, are substantially free of fat. As used herein “substantially free of fat” refers to nutritional compositions containing less than 0.5%, including less than 0.1% fat by weight of the total composition. “Substantially free of fat” also may refer to nutritional compositions disclosed herein that contain no fat, i.e., zero fat. Furthermore, embodiments of liquid nutritional compositions that have a desired acidic pH in the range of 2 to 5, e.g., juices, fruit juices, fruit-flavored beverages, etc., typically are substantially free of fat. Liquid nutritional compositions that are both clear and have a pH ranging 2 to 5 are also typically substantially free of fat. In certain exemplary embodiments, the pH of the nutritional composition may be 2.5 to 4.6. In other exemplary embodiments, the pH of the nutritional composition may be 3 to 3.5. In those embodiments of the nutritional compositions that are substantially free of fat but have some amount of fat present, the fat may be present as a result of being inherently present in another ingredient (e.g., a source of protein) or may be present as a result of being added as one or more separate sources of fat.

In certain exemplary embodiments disclosed herein, the nutritional composition may further comprise other optional components or ingredients that may modify the physical, chemical, aesthetic or processing characteristics of the nutritional composition or serve as pharmaceutical or additional nutritional components. Many such optional ingredients are known or otherwise suitable for use in medical food or other nutritional compositions or pharmaceutical dosage forms and may also be used in the nutritional compositions disclosed herein, provided that such optional ingredients are safe for oral administration and are compatible with the essential and other ingredients in the selected composition form.

Non-limiting examples of such optional ingredients include preservatives, emulsifying agents, buffers, fructooligosaccharides, galactooligosaccharides, polydextrose, and other prebiotics, probiotics, pharmaceutical actives, anti-inflammatory agents, additional nutrients, colorants, flavors, thickening agents and stabilizers, emulsifying agents, lubricants, and so forth.

In certain exemplary embodiments disclosed herein, the nutritional composition may further comprise at least one sweetening agent. Non-limiting examples of the at least one sweetening agent include at least one sugar alcohol such as maltitol, erythritol, sorbitol, xylitol, mannitol, isolmalt, and lactitol, or at least one artificial or high potency sweetener such as acesulfame K, aspartame, sucralose, saccharin, stevia, monk fruit, tagatose, or combinations thereof. The sweetening agents, especially as a combination of a sugar alcohol and an artificial sweetener, are especially useful in formulating liquid nutritional compositions having a desirable flavor profile. These sweetener combinations are especially effective in masking undesirable flavors, for example, as sometimes associated with the addition of vegetable proteins to a liquid nutritional composition. In certain exemplary embodiments disclosed herein, the nutritional composition may comprise at least one sugar alcohol with a concentration in a range from at least 0.01%, including from about 0.1% to about 10%, and also including from about 1% to about 6%, by weight of the nutritional composition.

A flowing agent or anti-caking agent may be included in certain exemplary embodiments of the nutritional composition to retard clumping or caking of a nutritional composition (when in the form of a powder) over time and to make the nutritional composition flow easily from its container. Any known flowing or anti-caking agents that are known or otherwise suitable for use in a nutritional powder or composition form are suitable for use herein, non-limiting examples of which include tricalcium phosphate, silicates, and combinations thereof. The concentration of the flowing agent or anti-caking agent in certain exemplary embodiments of the nutritional composition varies depending upon the composition form, the other selected ingredients, the desired flow properties, and so forth, but most typically range from about 0.1% to about 4%, including from about 0.5% to about 2%, by weight of the nutritional composition.

In certain other exemplary embodiments disclosed herein, the nutritional composition may further comprise any of a variety of, or combination of, vitamins or related nutrients, non-limiting examples of which include vitamin A, vitamin E, vitamin A palmitate, vitamin E acetate, vitamin C palmitate (ascorbyl palmitate), vitamin K, thiamine, riboflavin, pyridoxine, vitamin B₁₂, carotenoids (e.g., beta-carotene, zeaxanthin, lutein, lycopene), niacin, folic acid, pantothenic acid, biotin, vitamin C, choline, inositol, salts and derivatives thereof. In yet other exemplary embodiments disclosed herein, the nutritional composition comprises any of a variety of, or combination of, additional minerals, non-limiting examples of which include calcium, selenium, potassium, iodine, phosphorus, magnesium, iron, zinc, manganese, copper, sodium, molybdenum, chromium, chloride.

In certain exemplary embodiments disclosed herein, the nutritional compositions optionally include one or more masking agents to reduce or otherwise obscure the development of any residual bitter flavors and after taste in the nutritional compositions over time. Suitable masking agents include natural and artificial sweeteners, sodium sources such as sodium chloride, and hydrocolloids, such as guar gum, xanthan gum, carrageenan, gellan gum, and combinations thereof. The amount of masking agent in certain exemplary embodiments of the nutritional composition may vary depending upon the particular masking agent selected, other ingredients in the formulation, and other formulation or product target variables. Such amounts, however, most typically range from 0.1% to 3%, including 0.2% to 2.5%, by weight of the nutritional composition.

The various exemplary embodiments of the nutritional compositions disclosed herein may be prepared by any process or suitable method (now known or known in the future) for making a selected composition form, such as a nutritional solid, a nutritional powder, or a nutritional liquid. Many such techniques are known for any given composition form such as nutritional liquids or nutritional powders and can easily be applied by one of ordinary skill in the art to the various embodiments of the nutritional composition disclosed herein.

In one suitable manufacturing process for liquid nutritional compositions, for example, at least three separate slurries are prepared, including a protein-in-fat (PIF) slurry, a carbohydrate-mineral (CHO-MN) slurry, and a protein-in-water (PIW) slurry. The PIF slurry is formed by heating and mixing an oil (e.g., canola oil, corn oil, etc.) and then adding an emulsifier (e.g., lecithin), fat soluble vitamins, and a portion of the total protein (e.g., milk protein concentrate, etc.) with continued heat and agitation. The CHO-MN slurry is formed by adding with heated agitation to water: minerals (e.g., potassium citrate, dipotassium phosphate, sodium citrate, etc.), trace and ultra trace minerals (TM/UTM premix), thickening or suspending agents (e.g., avicel, gellan, carrageenan). The resulting CHO-MIN slurry is held for 10 minutes with continued heat and agitation before adding additional minerals (e.g., potassium chloride, magnesium carbonate, potassium iodide, etc.), or carbohydrates (e.g., fructooligosaccharide, sucrose, corn syrup, etc.), or combinations thereof. The PIW slurry is then formed by mixing with heat and agitation the remaining protein.

In accordance with this process, the three resulting slurries are blended together with heated agitation and the pH adjusted to the desired range, e.g., 6.6 to 7, after which the nutritional composition is subjected to high-temperature short-time (HTST) processing. The nutritional composition is heat treated, emulsified, homogenized, and cooled during HTST. Water soluble vitamins and ascorbic acid are added (if applicable), the pH is again adjusted (if necessary), flavors are added, and any additional water can be added to adjust the solids content to the desired range. At this point, the liquid nutritional composition may be packaged and sterilized according to any suitable sterilization technique, such as aseptic, retort, or hot-fill sterilization.

A nutritional powder, such as a spray dried nutritional powder or dry mixed nutritional powder, may be prepared by any collection of known or otherwise effective technique, suitable for making and formulating a nutritional powder. For example, when the nutritional powder is a spray dried nutritional powder, the spray drying step may likewise include any spray drying technique that is known for or otherwise suitable for use in the production of nutritional powders. Many different spray drying methods and techniques are known for use in the nutrition field, all of which are suitable for use in the manufacture of the spray dried nutritional powders herein.

One method of preparing the spray dried nutritional powder comprises forming and homogenizing an aqueous slurry or liquid comprising predigested fat, and optionally protein, carbohydrate, and other sources of fat, and then spray drying the slurry or liquid to produce a spray dried nutritional powder. The method may further comprise the step of spray drying, dry mixing, or otherwise adding additional nutritional or functional ingredients, including any one or more of the ingredients described herein, to the spray dried nutritional powder.

Other suitable methods for making nutritional compositions are described, for example, in U.S. Pat. No. 6,365,218 (Borschel, et al.), U.S. Pat. No. 6,589,576 (Borschel, et al.), U.S. Pat. No. 6,306,908 (Carlson, et al.), U.S. Pat. Appl. No. 20030118703 A1 (Nguyen, et al.), which descriptions are incorporated herein by reference to the extent that they are consistent herewith.

### EXAMPLES

The following examples illustrate certain embodiments or features of the nutritional compositions and methods and according to certain exemplary embodiments disclosed herein. The examples are given solely for the purpose of illustration and are not to be construed as limitations of the present disclosure, as many variations thereof are possible without departing from the spirit and scope of the disclosure.

### Example 1

Example 1 illustrates a nutritional composition according to one exemplary embodiment. All ingredient amounts listed in Table 1 are listed as kilogram per 1000 kg batch of product, unless otherwise indicated. Table 1 shows an exemplary formulation of a emulsion-type liquid nutritional composition containing fat, protein, and carbohydrates and has a pH in the range of 6.6-7. Assuming a density of 1.075 g/mL and a serving size of about 237 mL (about 8 fl. oz.), a nutritional composition according to the formulation shown in Table 1 has about 177 mg of EGCg per serving and about 1.5 g of HMB per serving. In addition, the nutritional composition includes 11 g of protein per serving (or about 0.047 g/mL), 40 g of carbohydrate per serving (or about 0.17 g/mL), and 6 g of fat per serving (or about 0.24 g/mL).

TABLE 1

| INGREDIENTS | Amount (kg/1000 kg) |
|---|---|
| Water | Quantity Sufficient |
| EGCg-containing Green Tea Extract¹ | 1.390 |
| HMB | 6.7 |
| Sucrose | 89.1 |
| Maltodextrin | 69.1 |
| Milk Protein Concentrate | 38.6 |
| Soy Oil | 13.3 |
| Canola Oil | 5.3 |
| Soy Protein Concentrate | 4.7 |
| Corn Oil | 4.1 |
| Potassium Citrate | 2.7 |
| Natural and artificial Vanilla Flavor | 2.0 |
| Magnesium Phosphate Dibasic | 1.9 |
| Sodium Citrate | 1.6 |
| Soy Lecithin | 1.4 |
| Tricalcium Phosphate | 1.3 |
| Magnesium Chloride | 1.2 |
| Sodium Chloride | 0.718 |
| Choline Chloride | 0.480 |
| Ascorbic Acid | 0.469 |
| Carrageenan | 0.450 |
| Ultra Trace Mineral/Trace Mineral Premix | 0.364 |
| Potassium Hydroxide (Processing aid) | 0.323 |
| Potassium Chloride | 0.308 |
| Vitamin Premix² | 0.1465 |
| Potassium Iodide | 0.000207 |

¹SUNPHENON ® 90D (available from Taiyo International, Inc. of Minneapolis, Minnesota) is a green tea extract that contains approximately 50% by weight of EGCg, i.e., 1.390 kg of green tea extract contains approximately 0.695 kg EGCg.
²Vitamin premix includes one or more of the following: dl-Alpha-Tocopheryl Acetate, Vitamin A Palmitate, Phylloquinone, Vitamin D3, Niacinamide, d-Calcium Pantothenate, Thiamine Chloride Hydrochloride, Pyridoxine Hydrochloride, Riboflavin, Folic Acid, Biotin, Cyanocobalamin, etc.

### Example 2

Example 2 illustrates a nutritional composition according to one exemplary embodiment. All ingredient amounts listed in Table 2 are listed as kilogram per 1000 kg batch of product, unless otherwise indicated. Table 2 shows an exemplary formulation of a clear-type liquid nutritional composition that is substantially free of fat and having a pH in the range of 3-3.5. Assuming a density of 1.05 g/mL and a serving size of about 296 mL (about 10 fl. oz.), a nutritional composition made according to the formulation shown in Table 2 has about 188 mg of EGCg per serving and about 1.5 g of HMB per serving. In addition, the nutritional composition includes 9 g of protein per serving (or about 0.0304 g/mL), 35 g of carbohydrate per serving (or about 0.118 g/mL), 0 g of fat per serving, and an energy content of 180 kcal per serving (or about 0.61 kcal/mL).

TABLE 2

| INGREDIENTS | Amount (kg/1000 kg) |
|---|---|
| Water | Quantity Sufficient |
| Sucrose | 50.7 |
| Corn syrup solids | 61.3 |
| Acidified Whey Protein Isolate | 35.7 |
| Citric Acid | 2.00 |
| Flavoring | 2.00 |
| EGCg-containing Green Tea Extract¹ | 1.212 |
| HMB | 6.7 |
| Ascorbic Acid | 0.535 |
| Liquid Sucralose (25%) | 0.275 |
| Ultra Trace Mineral/Trace Mineral Premix | 0.230 |
| Vitamin Premix² | 0.219 |
| Acesulfame Potassium | 0.110 |
| Antifoam processing aid (non-silicone) | 0.060 |
| Coloring | 0.0589 |
| Natural and Artificial Peach Flavor | 2.0 |
| Folic Acid | 0.0013 |
| Potassium Iodide | 0.000204 |

¹SUNPHENON ® 90D, which is a green tea extract that contains approximately 50% by weight of EGCg, i.e., 1.212 kg of green tea extract contains approximately 0.606 kg EGCg.
²Vitamin premix includes one or more of the following: dl-Alpha-Tocopheryl Acetate, Vitamin A Palmitate, Phylloquinone, Vitamin D3, Niacinamide, d-Calcium Pantothenate, Thiamine Chloride Hydrochloride, Pyridoxine Hydrochloride, Riboflavin, Folic Acid, Biotin, Cyanocobalamin, etc.

### Example 3

Example 3 illustrates the effect of 8 weeks of dietary supplementation in the aged Sprague Dawley (SD) rat model of sarcopenia. More particularly, the level of intramuscular FGF2 in gastrocnemius muscle lysates of aged SD rats (21 month) were analyzed and compared. The four experimental groups included:

1) control group: fed AIN-93M (purified diet);

2) EGCg group: fed AIN-93M+EGCg (Teavigo®-DSM-50 mg/kg bw);

3) HMB group: fed AIN-93M+Ca-HMB (340 mg/kg bw); and

4) HMB+EGCg group: fed AIN-93M+Ca-HMB (340 mg/kg bw)+EGCg (Teavigo®-50 mg/kg bw).

A total of 40 rats were divided equally among the four groups. Whole gastrocnemius muscles were collected from euthanized rats and flash frozen in liquid nitrogen. Approximately 200 mg tissue was obtained from each muscle by mechanical biopsy punch. Specimens were then pulverized on liquid nitrogen and transferred to 15 mL conical tube and weighed. A mild, detergent free lysis buffer (9×) was added to the dried specimen in the conical tube. 9× Lysis buffer (50 mM Tris.HCl, 2 mM EDTA, pH 7.4/NaOH) was supplemented with a mammalian protease inhibitor cocktail (Sigma) at a 1:200 dilution and stored on ice. The specimens were then vortexed again for 10 sec. and transferred to a 1.5 mL tube. Lysates were centrifuged/clarified for 5 min. at 11000 rpm at 4° C. in a standard table-top Eppendorf centrifuge. Supernatant (1 mL) was aliquotted to a 2 mL cryotube and stored at −80° C. Lystates were analyzed by Myriad Rules Based Medicine (Austin, Tex.) on the RodentMAP® v. 2.0 Antigens and Rat METABOLIC® multi-analyte profiling platforms. Table 3 shows the levels of intramuscular FGF2 (ng/mL of lysate) for the rats at the end of the 8 week study.

TABLE 3

| | Control | EGCg | HMB | EGCg + HMB |
|---|---|---|---|---|
| Rat 1 | 3.7 | 5.5 | 6.1 | 3.7 |
| Rat 2 | 3.7 | 3.1 | 3.1 | 4.9 |
| Rat 3 | 3.1 | 3.7 | 4.3 | 4.9 |
| Rat 4 | 11.0 | 7.8 | 11.0 | 3.1 |
| Rat 5 | 3.1 | 3.1 | 4.0 | 3.7 |
| Rat 6 | 4.3 | 3.1 | 4.9 | 9.9 |
| Rat 7 | 5.5 | 3.7 | 15.0 | 3.7 |
| Rat 8 | 6.6 | 16.0 | 21.0 | 4.3 |
| Rat 9 | 16.0 | 15.0 | n/a | 4.9 |
| Rat 10 | 16.0 | 9.9 | n/a | n/a |
| AVE | 7.3 | 7.09 | 8.68 | 4.79 |
| SEM | 1.63 | 1.58 | 2.28 | 0.68 |
| % Change from | | −2.9 | 18.8 | −34.4 |
| Control | | | | |
| TTEST | | | | 0.19 |

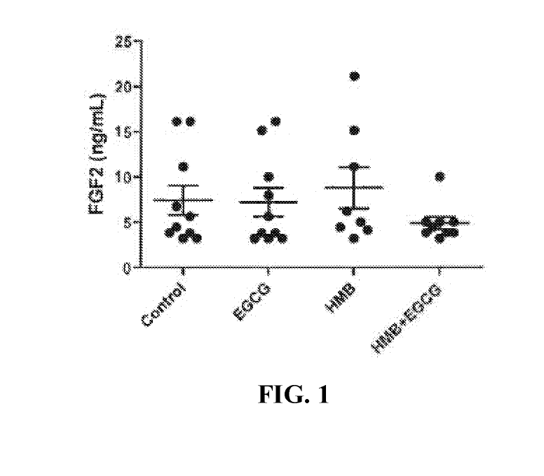

FIG. 1 is a graph illustrating the data shown in Table 3. Table 3 shows a decrease in the intramuscular level of FGF2 for the EGCg+HMB group of 34%. This substantial decrease was not observed in any of the other treatment groups. Thus, as disclosed herein, the combination of EGCg and HMB decreases the level of intramuscular FGF2 greater than expected from the values of either the EGCg or the HMB group alone.

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use. See Bryan A. Garner, A Dictionary of Modern Legal Usage 624 (2d. Ed. 1995). Also, to the extent that the terms “in” or “into” are used in the specification or the claims, it is intended to additionally mean “on” or “onto.”

While the present application has been illustrated by the description of embodiments thereof, and while the embodiments have been described in considerable detail, it is not the intention of the applicants to restrict or in any way limit the scope of the appended claims to such detail. Additional advantages and modifications will readily appear to those skilled in the art. Therefore, the application, in its broader aspects, is not limited to the specific details, the representative compositions and processes, and illustrative examples shown and described. Accordingly, departures may be made from such details without departing from the spirit or scope of the general inventive concepts.

1. A nutritional composition for enhancing the regenerative capacity of muscle in an individual, the nutritional composition comprising:
a source of EGCg in an amount sufficient to provide 0.1 to 1 gram of EGCg per serving of the nutritional composition; and
a source of beta-hydroxy-beta-methylbutyrate (HMB) in an amount sufficient to provide 0.5 to 3 grams of HMB per serving of the nutritional composition.

2. The nutritional composition of claim 1, further comprising at least one of: a source of protein, a source of carbohydrate, a source of fat, and combinations thereof.

3. The nutritional composition of claim 1, wherein the nutritional composition comprises protein in an amount sufficient to provide 5 to 50 grams of protein per serving of the nutritional composition.

4. The nutritional composition of claim 2, wherein the nutritional composition is a liquid nutritional composition.

5. The nutritional composition of claim 4, wherein the nutritional composition has a pH of 6 to 9.

6. The nutritional composition of claim 2, wherein the nutritional composition comprises: a source of carbohydrate, a source of protein, and is a clear liquid nutritional composition with a pH of 2 to 5.

7. The nutritional composition of claim 2, wherein the nutritional composition is a solid nutritional composition.

8. The nutritional composition of claim 1, further comprising at least one branched-chain amino acid selected from the group comprising: leucine, isoleucine, valine, alpha-ketoisocaproic acid and alpha-hydroxyisocaproic acid, alpha-ketoisovaleric acid, alpha-hydroxyisovaleric acid, beta-hydroxyisobutyric acid, 2-oxo-3-methylvaleric acid, 2-hydroxy-3-methylvaleric acid, 3-hydroxy-2-ethylpropionic acid, 3-hydroxy-2-methylbutyric acid, and combinations thereof.

9. A method for enhancing the regenerative capacity of muscle in an elderly individual, the method comprising:
administering to an elderly individual, a composition comprising:
a therapeutically effective amount of epigallocatechin-gallate (EGCg); and
a therapeutically effective amount of beta-hydroxy-beta-methylbutyrate (HMB);
whereby the administration results in an enhancement of regenerative capacity of the elderly individual's muscle(s).

10. The method of claim 9, wherein the enhancement is within skeletal muscle.

11. The method of claim 9, wherein the elderly individual is administered HMB in an amount sufficient to provide 0.5 to 4 grams of HMB per day; and EGCg in an amount sufficient to provide 0.1 to 1.5 grams of EGCg per day.

12. The method of claim 9, wherein the elderly individual is administered the composition for 2 months or more.

13. The method of claim 9, wherein the composition is administered daily.

14. The method of claim 9, wherein the composition is administered as part of a nutritional composition comprising at least one of: a source of protein, a source of carbohydrate, a source of fat, and combinations thereof.

15. The method of claim 9, wherein the administration results in a decrease in the intramuscular level of fibroblast growth factor (FGF2) of the elderly individual.

16. The method of claim 15, wherein the intramuscular level of FGF2 is decreased by between 1 and 50% compared to the level of FGF2 prior to initiating the method.

17. A method for decreasing the level of fibroblast growth factor (FGF2) in the muscle of an elderly individual, the method comprising:
administering a composition comprising a therapeutically effective amount of epigallocatechin-gallate (EGCg) and a therapeutically effective amount of beta-hydroxy-beta-methylbutyrate (HMB) to an elderly individual, wherein the HMB and EGCg are present in the composition in a weight ratio between 1:2 to 30:1;
whereby the administration results in a decrease in the intramuscular level of FGF2 of the elderly individual.

18. The method of claim 17, wherein the composition is administered as part of a nutritional composition comprising at least one of: a source of protein, a source of carbohydrate, a source of fat, and combinations thereof.

19. The method according to claim 17, wherein the intramuscular level of FGF2 is decreased by between 1 and 50% compared to the level of FGF2 prior to initiating method.

20. (canceled)

21. The nutritional composition of claim 1, wherein the individual suffers from at least one of sarcopenia, cachexia, chronic obstructive pulmonary disease (COPD), end stage renal disease (ESRD), and acquired immune deficiency syndrome (AIDS); or the individual is bedridden or otherwise immobile and suffers from muscle disuse; or the individual is undergoing rehabilitation subsequent to a period of injury, disease, surgery, immobilization, hospitalization, or combinations thereof.
